(19) United States
(12) Patent Application Publication
(10) Pub. No.: US 2014/0316254 A1
(43) Pub. Date: Oct. 23, 2014

(54) APPARATUS AND METHODS FOR OPTICAL POSITION SENSING

(71) Applicant: CLPH, LLC, Palo Alto, CA (US)

(72) Inventors: Christian S. Eversull, Palo Alto, CA (US); Stephen A. Leeflang, Sunnyvale, CA (US); Christopher E. Woods, Stanford, CA (US)

(21) Appl. No.: 14/217,264

(22) Filed: Mar. 17, 2014

Related U.S. Application Data
(60) Provisional application No. 61/800,229, filed on Mar. 15, 2013.

(51) Int. Cl. A61B 5/06

(52) U.S. Cl. CPC A61B 5/065
USPC 600/424

(57) ABSTRACT

Apparatus, systems, and methods are provided for optically sensing position within body lumens within a patient's body, e.g., in blood-filled vessels and chambers. In one embodiment, the apparatus includes a tubular member including a proximal end, a distal end sized for introduction into a patient's body, and one or more lumens extending between the proximal and distal ends; a distal tip on the distal end for contacting tissue; and one or more optical elements on the distal tip configured to transmit illumination beyond the distal tip and capture optical signals from tissue or fluids adjacent the distal tip. The distal tip may be positioned within a body lumen, and the optical elements may be used to detect the proximity of the distal tip relative to tissue adjacent the body lumen.

This application claims benefit of co-pending provisional application Ser. Nos. 61/800,229, filed Mar. 15, 2013 the entire disclosure of which is expressly incorporated by reference herein.

### FIELD OF THE INVENTION

The present invention relates generally to apparatus and methods for performing medical procedures, and, more particularly, to devices, systems, and methods for optically sensing position in body lumens, such as blood-filled vessels and chambers.

### BACKGROUND

In cardiac ablation cases for the treatment of arrhythmias (and/or other cardiac electrical propagation problems), such as atrial fibrillation, the efficacy of ablation (e.g., radiofrequency (RF), cryoablation, and the like) is a function of the quality of contact of the ablation probe with the tissue. Good contact (contact with good apposition to the heart tissue) results in effective lesions that block propagation of unwanted electrical signals, while poor contact may result in ineffective lesions that do not adequately block unwanted electrical signal propagation.

Historically, physicians have relied on a number of indirect methods of evaluating and improving the odds of good tissue contact. These include tactile feedback, temperature sensing, and impedance measurements, which methods are frequently used in conjunction with imaging modalities such as traditional fluoroscopy and/or electromechanical navigation systems. These methods and tools, although helpful, have proven insufficient to evaluate and ensure the level of apposition required for effective ablation lesions.

To this end, recent developments have been directed toward catheters that include one or more mechanisms for measuring force at the tip. These mechanisms include mechanical, electrical, and/or other force sensing mechanisms, which may include multi-axis force sensing, e.g., ability to measure in force in x, y, and/or z direction(s) relative to the catheter tip. As the catheter tip is pressed against tissue, a force measurement is generated. A correlation is then made between force and quality of tissue apposition, and thus quality of an ablation lesion created at that location.

In spite of these most recent improvements, there are still significant problems that remain. At relatively low contact angles (e.g., side apposition), it may be difficult to determine with adequate precision what portion of the electrode is in contact with the tissue and thus difficult to determine the desired apposition force for ideal ablation. A lower angle means more contact of tissue with the electrode, which means a higher apposition force is required to have the same degree of apposition pressure. Additionally, presently used mechanisms consume a large portion of the device profile and take away or limit performance in other important areas including, for example, profile, irrigation, flexibility, number of electrodes, and the like. Thus, an improved system that can report tissue proximity and/or degree of apposition while addressing these limitations is of significant value.

### SUMMARY

The present invention is directed to apparatus and methods for performing medical procedures including optically sensing tissue physiology and other characteristics. More particularly, the present invention is directed to devices, systems, and methods for optically sensing tissue proximity in body lumens, such as blood-filled vessels and chambers.

In accordance with one embodiment, an apparatus is provided for performing a procedure within a patient's body that includes a tubular member comprising a proximal end, a distal end sized for introduction into a patient's body, and one or more lumens extending between the proximal and distal ends; a distal tip on the distal end for contacting tissue; one or more optical elements on the distal tip configured to transmit illumination beyond the distal tip and capture optical signals from tissue or fluids adjacent the distal tip.

In accordance with another embodiment, a method is provided for performing a procedure within a patient's body that includes introducing a distal end of a tubular member into a patient's body; placing a distal tip within a body lumen of the patient's body in contact with or in proximity to tissue adjacent the body lumen; and using one or more optical elements on the distal tip to detect the proximity of the distal tip relative to the tissue. For example, illumination may be directed from the distal tip towards the tissue, and optical signals may be acquired corresponding to light reflect towards the distal tip within the body lumen, e.g., from the tissue and/or fluid within the body lumen, and the optical signals may be analyzed to determine the proximity of the distal tip relative to the tissue.

In an exemplary embodiment, the body lumen may be a chamber of the patient's heart, and the optical signals may be analyzed to create an electro-anatomical model of the patient's heart, e.g., using the optical signals to detect the contraction of tissue within the heart at discrete locations in time and space.

Other aspects and features including the need for and use of the present invention will become apparent from consideration of the following description taken in conjunction with the accompanying drawings.

### BRIEF DESCRIPTION OF THE DRAWINGS

It will be appreciated that the exemplary apparatus shown in the drawings are not necessarily drawn to scale, with emphasis instead being placed on illustrating the various aspects and features of the illustrated embodiments. The drawings illustrate exemplary embodiments of the invention, in which:

FIG. 1 is a perspective view of a catheter including a handle on a proximal end and an electrode tip with optical sensors on a distal end thereof.

FIG. 1A is a perspective detail showing an exemplary embodiment of a tip including a single combination illumination and capturing element.

FIG. 1B is a perspective detail showing another exemplary embodiment of a tip including split illumination and capturing elements.

FIG. 2A is schematic view of an exemplary embodiment of a hardware set-up including a dichroic element for a combination illumination and capturing element.

FIG. 2B is a schematic view of another exemplary embodiment of a hardware set-up including components configured for split illumination and capturing elements.

FIG. 3 is a cross-sectional view of a body lumen, showing a distal end of a treatment catheter positioned within the body lumen and a distal tip thereof interacting with body tissue adjacent the body lumen.

FIG. 4 is a graph showing a relationship between light transmission and distance in a semi-opaque fluid for a given wavelength.

### DETAILED DESCRIPTION OF THE EXEMPLARY EMBODIMENTS

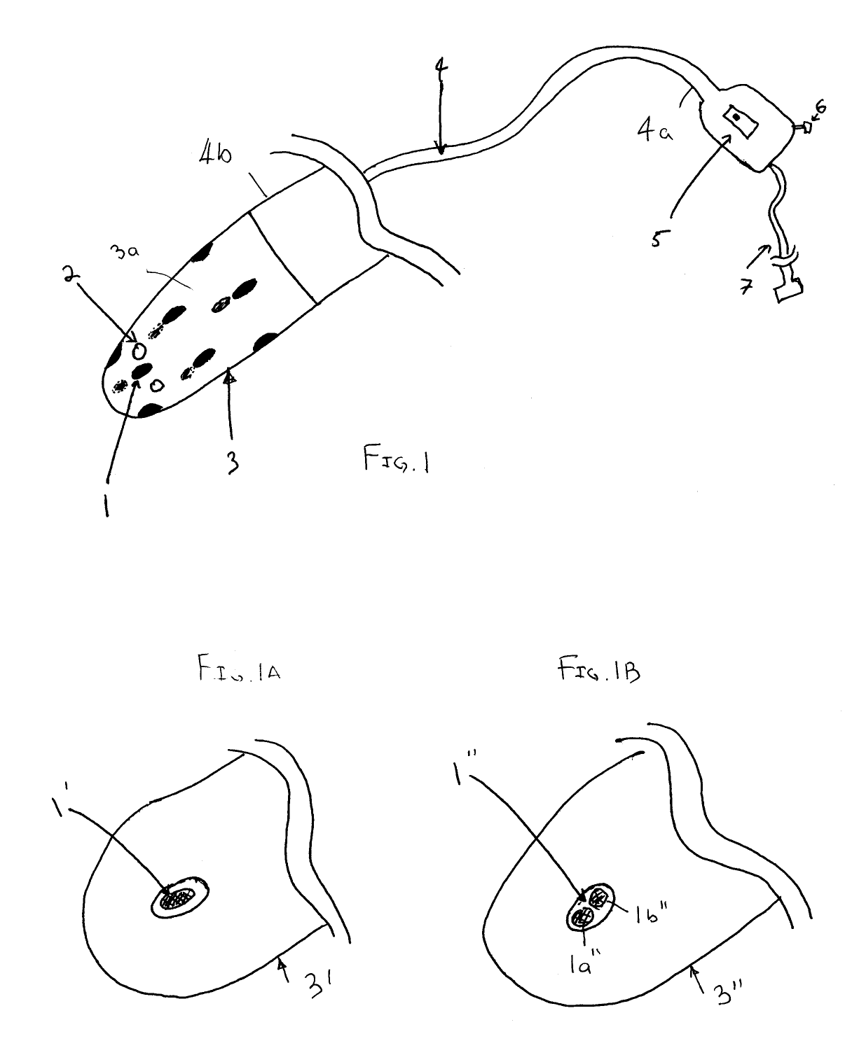

Turning to the drawings, FIG. 1 shows an exemplary embodiment of a diagnostic or treatment catheter 4 including a proximal end 4a, e.g., including a handle or hub 5, a distal 4b end sized for introduction into a patient's body, and a distal tip 3, e.g., including a conductive electrode, e.g., for sensing and/or ablation. As described elsewhere herein, the distal tip 3 may be configured for sensing proximity to tissue via one or more optical elements or sensors 1, which may be capable of at least one of providing illumination or other optical output and detecting an optical signal. The catheter 4 may be included in a system for performing one or more desired procedures, e.g., a diagnostic or therapeutic procedure, that includes other components, e.g., one or more illumination sources, fluid source, aspiration sources, external processors, displays, and/or user interfaces (not shown).

In the exemplary embodiment, the distal tip 3 includes a plurality of optical elements 1 located at various locations about an outer surface 3a thereof. The locations of the optical elements 1 may be optimized according to specific anatomy likely to be encountered and/or particular diagnostic and/or therapeutic applications. Further, the number of and spacing between the elements 1 may vary with the size and/or aspect ratio of the distal tip 3, e.g., based on the intended target disease, indication, anatomy, and/or therapy to be delivered.

In the exemplary embodiment illustrated in FIG. 1, three circumferential arrays or sets of optical elements 1 are depicted on a tip 3 providing a single diagnostic and/or therapeutic electrode. Alternatively, if desired, multiple spaced apart electrodes (not shown) may be provided on the distal end 4a and each electrode may include one or more optical elements, e.g., one or more circumferential arrays, similar to those shown in FIG. 1.

As shown, if desired, these elements 1 may be staggered, e.g., to increase uniformity of distribution over the outer surface 3a of the distal tip 3. Relatively shorter length distal tips 3 may have fewer optical elements 1 and/or the elements 1 be concentrated toward the distal-most end of the tip 3, while relatively longer tips 3 may afford room for and/or require more optical elements, for example, in order to detect when the entire length of the distal tip 3 is in proximity to and/or in contact with tissue. The may be useful, for example, when performing RF ablation with a catheter having an eight millimeter (8 mm) or longer electrode.

Some applications my demand less resolution and consequently fewer optical elements 1. For example, when simply detecting tissue contact or tissue thickness before obtaining a tissue sample, e.g., for example in biopsy applications. In some cases, the optical elements 1 may be concentrated more to one side or the other, e.g., relative to a shape or deflection plane of the distal end 4b, such as when a particular portion of the target anatomy will always touch a limited portion of the electrode(s). The catheter tip 3 may include various other features, such as one or more irrigation holes or ports 2 (two shown in FIG. 1), e.g., for cooling; one or more radiopaque features for fluoroscopic or other visualization; one or more magnetic elements, e.g., for navigation and/or manipulation; and/or other features known in the art to be useful on catheter devices.

Turning to FIG. 1A, an alternative embodiment of a distal tip 3′ is shown that may be provided on the catheter 4 of FIG. 1, and that includes a single optical element 1′ capable of both generating/delivering illumination and capturing/sensing an optical signal. For example, the optical element 1 may emit light at a predetermined wavelength and sense an electromagnetic signal reflected and/or back-scattered from tissue. Alternatively, the optical element 1′ may emit light at a pre-determined wavelength and sense an electromagnetic signal emitted by tissue in response to the emitted light, e.g., based on tissue auto-fluorescence. The optical element 1′ may include one or more fiber optic elements, e.g. glass or plastic fibers with cladding (not shown), and may further include one or more lenses (also not shown) coupled to proximal and/or distal ends of the fiber(s), e.g., to focus and/or otherwise direct light passing therethrough. Optionally, a prism or other structure (not shown) may be provided within the distal tip 3, e.g., to direct light from a distal end of fiber(s) extending substantially axially along the catheter 4 to a transverse angle, e.g., substantially perpendicular relative to the longitudinal axis of the catheter 4.

FIG. 1B depicts another alternative embodiment of a distal tip 3″ that may be provided on the catheter 4 of FIG. 1, and that includes an optical element 1″ in which illuminating and sensing components are separated. For example, an illumination component 1a″ may include one or more glass or plastic fibers with optional proximal and/or distal lenses capable of carrying illumination from a source (not shown) at the proximal end 4a of the catheter 4 to the distal tip 3.″ Alternatively, the illumination component 1a″ may include a light emitting diode (LED) or other emitter mounted in or otherwise to the distal tip 3 capable of generating optical emissions.

Similarly, a sensing component 1b″ may include one or more glass or plastic fibers with optional proximal and/or distal lenses capable of conveying optical signals from the distal tip 3 to a sensor (not shown), e.g. a CCD, CMOS, photodiode, and the like, at the proximal end 4a of the catheter 4, e.g., within the handle 5. Alternatively, the sensing component 1b″ may include a photodiode or other sensor, e.g., CCD, CMOS, and the like, mounted in or otherwise to the distal tip 3 and capable of directly detecting an optical signal. In an exemplary embodiment, the sensing component may include a high speed camera configured to capture images at speeds greater than one hundred frames per second. Various other spatial arrangements (not shown) of separated emitting and sensing elements may be provided, e.g., a central sensing element surrounded by an annular illumination element. Such a configuration may be constructed using a bundle of individual fiber optics or an optically transmissive core separated from an optically transmissive annular jacket, e.g., by reflective cladding to substantially isolate the two components.

Where one or more fiber optics are used, it should be noted that individual fibers, e.g., glass, plastic, and the like, may be quite small, for example, having a diameter or other cross-section between about twenty and one hundred microns (20-100 μm). Thus, many such fibers may be integrated into the distal tip 3 of a catheter 4, e.g., based on the number of elements and/or arrays desired for a particular application. Alternatively, coherent fiber image bundles may be used which, while generally larger, e.g., between about 250 and 650 microns in diameter, allow for spatial resolution of a sensed signal and/or spatial segregation of emitted illumination.

With respect to illuminating light that may be used to detect tissue proximity and/or other tissue characteristics and/or aspects of tissue physiology, one or more wavelengths and/or spectra may be selected depending on the targeted tissue and/or the information desired. Illuminating light may include broad spectrum light, multiple narrow bands of spectra, or discrete bands of some wavelengths and broader portions of others. For gross proximity sensing relative to tissue, certain spectra of infrared illumination have improved abilities to penetrate blood and/or other tissue with less absorption and/or reflectance relative to shorter wavelength illumination. Thus, illumination light may be selected to preferentially pass through blood and reflect from cardiac or vascular tissue. An increase in intensity of reflected and/or backscattered light may be detected when approaching tissue through blood, and this intensity shift interpreted to determine proximity to tissue. Alternatively, relatively short wavelengths of light, e.g., light in the ultraviolet (UV) spectrum may be used to provide stark contrast between blood and tissue. For example, short wavelengths are generally rapidly scattered and/or absorbed by blood and better reflected and/or backscattered by tissue. Thus, very little reflected and/or or backscattered signal may be seen using a UV illumination source until all of the blood is displaced from the optical path and tissue is in direct apposition to the optical element 1. In an alternative embodiment, a visible light source may be used instead.

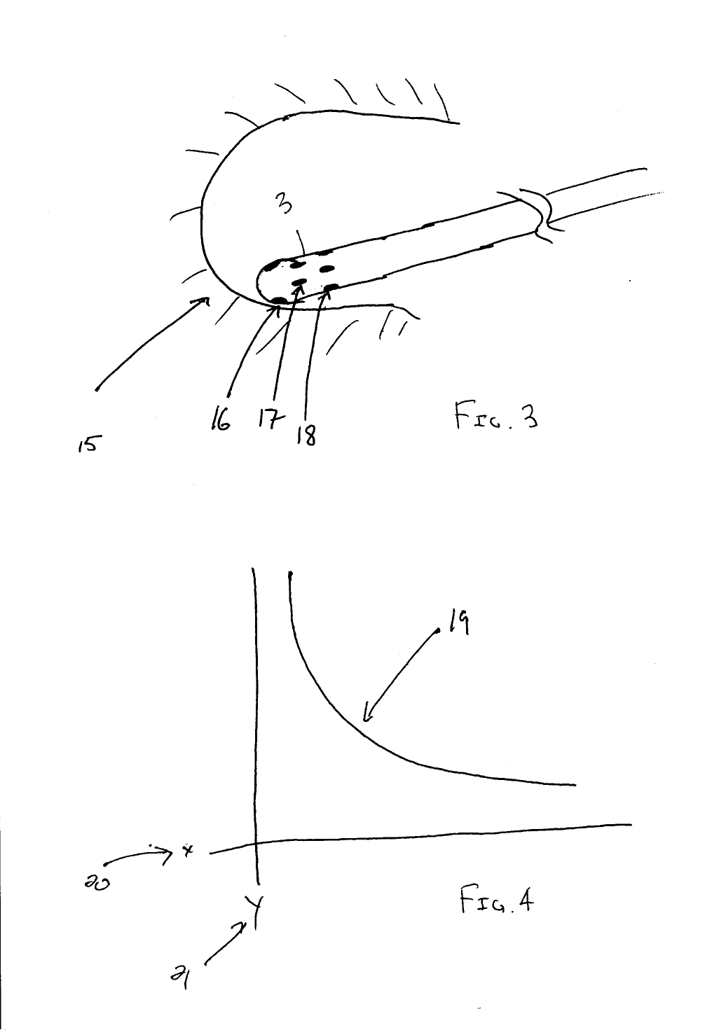

Turning to FIG. 3, the techniques described herein may be used to readily establish whether optical element 16 is in intimate contact with tissue 15, while optical element 18 is close to tissue 15 but not in intimate contact, e.g., having a small distance of intervening blood pool, and that optical element 17 is not directed toward tissue 15 of any relevant proximity. Within the setting of RF ablation, for example, this detailed proximity sensing may enable a physician to know one or both of whether to adjust and how to adjust a catheter to a position having optimal contact between an electrode tip 3 and tissue 15, e.g., in order to create a controlled lesion in the tissue 15. For example, the catheter may be manipulated until its distal tip 3 is more parallel to the tissue whereupon both elements 16 and 18 may report intimate tissue contact. This detailed determination of contact in terms of both proximity and angle is not possible with even sophisticated multi-axis force-sensing catheters as force does not necessarily relate directly to angle of approach and/or position of the catheter tip with respect to tissue.

FIG. 4 shows an exemplary curve 19 representing the relationship between transmission (y-axis) 21 as a function of depth (x-axis) 20 of a given wavelength of light through semi-opaque fluids, e.g., transmission of visible or UV light through blood. Transmission is generally attenuated proportional to the depth of the semi-opaque fluid. Thus, a detected signal of reflected and/or backscattered light may become attenuated as more fluid, e.g. blood, is present between the adjacent tissue and an optical emitter/sensor and this principal may be used to determine proximity to tissue of a distal tip 3 of a catheter (such as catheter 4 shown in FIG. 1), e.g., using one or more processors (not shown) within and/or otherwise coupled to the handle 5 and/or the optical elements.

Correspondingly, data from one or more optical sensors capable of determining tissue proximity/contact, and/or catheter position/angle relative to tissue may be used in real time to construct an animated model of the catheter 4 on a display (not shown), e.g., coupled to the one or more processors, allowing a user to view and use the model to guide movements and treatments using the catheter 4.

As noted above, an illumination source may emit UV light and/or other wavelengths known to cause auto-fluorescence of tissue. One or more specific spectra and/or wavelengths may be selected according to the specific tissue of interested. For example, a wavelength between about three hundred and four hundred thirty nanometers (300-430 nm) may be used to interrogate cardiac tissue. Detection of auto-fluorescence may be useful in a number of ways, including the ability to differentiate scar from normal cardiac tissue (e.g., collagen is known to be more highly auto-fluorescent than normal cardiac myocytes), fibrous from muscular tissue, and/or to evaluate real-time changes occurring in the tissue, e.g., in response to burning, freezing, and/or other forms of energy delivery such as those used for ablation. Other tissue conditions including structural, histologic, or physiologic may also be detected pre, during, or post treatment. For example,

In addition or alternatively, the techniques described herein may be used to detect one or more structural, anatomic, and/or physiologic features of tissue in addition to or alternatively to detecting proximity. For example, cardiac tissue may be illuminated in order to generate a reflected and/or back-scattered light signal, and/or light signal generated by auto-fluorescence. Compressed tissue, e.g., due to local contraction of a heart, may be more dense than relaxed tissue, and therefore may increase reflection of light and/or auto-fluorescence. Thus, the optical element(s) may used to detect and/or measure localized cardiac contraction, which contraction correlates directly with electrical activity in the heart (i.e., electro-anatomical coupling).

For example, the intensity of reflected, back-scattered, and/or auto-fluorescent light increases as cardiac muscle tissue contracts, e.g., as cells become smaller and the number and/or density of cells in an optical field increases. Contraction takes place in an organized fashion across the tissue of the heart and may be measured optically at a single point (e.g., by a single optical sensing element) or over a large field (e.g., using a camera, CCD array, CMOS array, and the like). Thus, localized contraction may be identified and correlated to local electrical activity, thereby allowing electrical modeling of a heart using multiple optical sensors or individual optical sensors moved along the wall of the heart in a desired manner.

For example, with reference to FIG. 3, if all elements 16, 17 and 18 were in contact with cardiac tissue, each may detect an intensity change or other change in optical signal produced by contraction in the cardiac tissue. In general, each element may detect a change in signal at a different point in time corresponding to the spatial propagation of electrical activity followed by contraction across the heart. By imaging many discrete points, e.g., using a coherent fiber bundle, camera, CCD array, CMOS array, and the like, propagation of contraction (correlating to electrical activity) across a large portion of the heart may be monitored, e.g., to detect normal conduction pathways, abnormal pathways, such as pathologic conduction channels through scar, various arrhythmias, and the like.

Restated, optical sensors may be used to identify beating of the heart in a precise location at a precise point in time. Similarly, multiple sensors may provide such information for many points across the heart. This information may be used to monitor the time, position, and/or intensity of contraction(s) throughout the heart. Using this approach, a model of contraction throughout the heart may be quickly, easily, and reliably created, which model may correspond directly to electrical activity of the heart. As noted above, electrical activity and cardiac tissue contraction are related by cause-and-effect in normal tissue. Likewise in scarred or otherwise damaged tissue, muted or absent contraction corresponds to muted or absent electrical activity. The systems and methods herein may use such differences to enhance the modeling.

Returning to FIG. 1, the handle 5 may include one or more connectors, such as connectors 6, 7, for coupling one or more devices to the catheter 4. For example, the connector(s) 6, 7 may include one or more luer connectors, electrical connectors, optical connectors for detectors, an illumination source, and the like. For example, a luer connector 7 may be provided for connecting a source of fluid and/or aspiration to the catheter 4, e.g., via the ports 2 in the distal tip 3 and an infusion/aspiration lumen (not shown) extending between the proximal and distal ends 4a, 4b of the catheter 4.

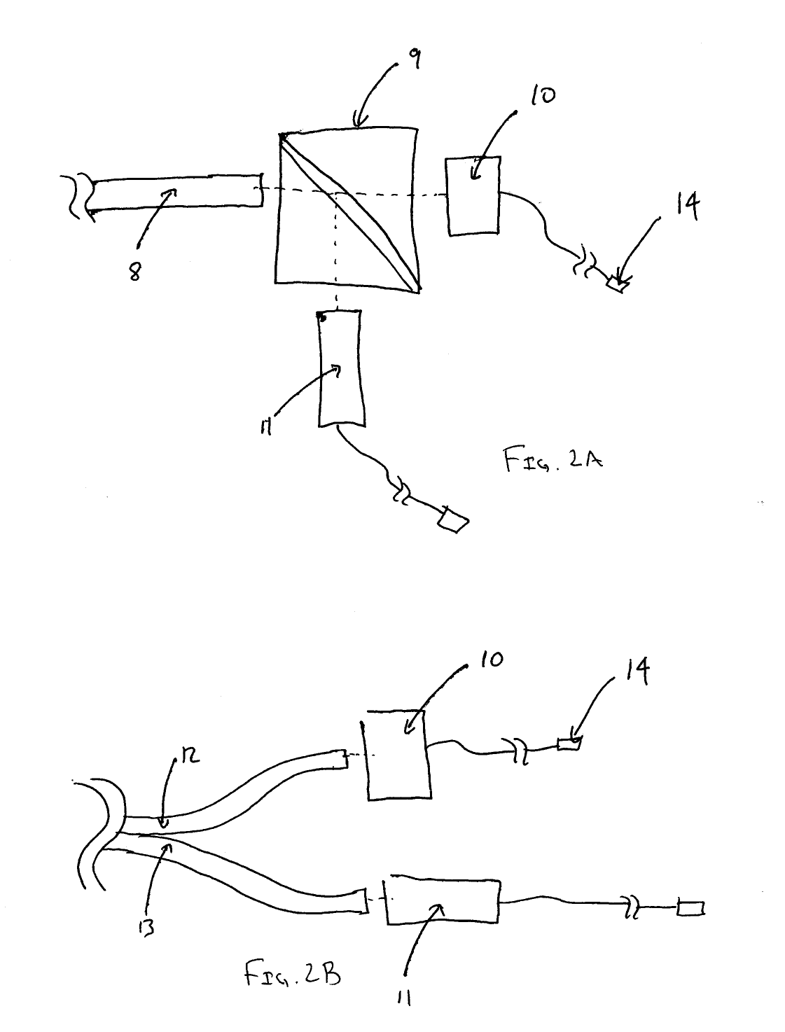

Turning to FIGS. 2A and 2B, schematics are shown of various elements that may be included in any of the apparatus and systems herein, such as the catheter 4 shown in FIG. 1 e.g., to provide excitation and/or illumination wavelengths and elements capable of sensing optical signals. The embodiment shown in FIG. 2A includes a dichroic element 9 that enables a optical pathway 8 that has passed through the length of the catheter 4 from the distal tip 3, as previously described, to both deliver illumination or excitation energy and to return an optical signal for analysis. An excitation/illumination source 11 (e.g., LED, light bulb, laser, and the like) and an optical sensor 10 (e.g., a photodiode, CCD, CMOS, and the like) with signal output 14 are also shown.

In another embodiments shown in FIG. 2B, separate optical pathways 12 and 13 are shown that may pass through the length of the catheter 4, one to coupled to an excitation/illumination source 11 and the other to an optical sensor 10 with signal output 14. As noted above, optical sensors 10 may include one or more photo-diodes, each coupled to one or more optical fibers. Additionally, multi-sensor arrays may be used. Image sensors such as CCD and CMOS may be used. In this case, one or a plurality of optical fibers may be coupled to a different portion of the CCD or CMOS sensor to separately evaluate hue, intensity, timing, and/or other parameters. Additionally, as mentioned previously, optical sensors may instead be located distally, e.g., at or near the distal tip 3 of the catheter 4. Excitation/illumination sources may include one or more LEDs, incandescent bulbs, lasers, and the like. Input and/or output light may be focused using lenses and/or filtered, as desired, to achieve enhance the desired signal.

With continued referent to FIGS. 2A and 2B, in an exemplary embodiment, the illumination/excitation source is located proximally, within or external and coupled to the handle 5, and transmitted distally through the optical pathway 8 or 13. Alternatively, the illumination/excitation source(s) may be located distally, e.g. at or near the distal tip 3, for example, using one or more LEDs. Optical pathways 8, 12, and 13 may include single mode, multi-mode, or coherent image fiber bundles composed of glass or glass-like elements and/or plastic fiber elements.

Additionally, the systems herein may include elements that while not specifically shown in the exemplary drawings, are helpful and/or are necessary to the proper function of the system in a wide range of intravascular, intra-luminal, and/or minimally invasive medical applications beyond cardiac ablations. These include one or more signal processors, user interfaces, navigable catheter features (such as steering or deflection elements), ablation sources/elements, and/or distal optical clearing elements (such as features to wipe or flush the sensing interface with tissue or blood, or other transparent guard or extender to prevent obscuring the signal).

With regards to signal processing, the systems herein may have the ability to gather multiple signals and multiple parameters. For example, a system may illuminate using one or more wavelengths and or ranges of wavelengths and may detect changes in wavelength and/or intensity and hue of collected light. Furthermore, a system may detect timing with respect to illumination and collection. Illumination may be continuous or pulsed. For example, an illumination signal may be pulsed and or alternate with one or more illumination signals, which may also be pulsed. Multiple signals/parameter may be used to determine proximity or other characteristics, such as those previously described, including determining heart beat and/or heartbeat timing, constructing a surrogate model of electrical activity, evaluating scar and ablated lesions, determining tissue thickness, ablation lesion depth, and the like.

Likewise, with regard to user interface, individual or composite output(s) of one or more sensors may be displayed, e.g., in an intuitive way to ensure the catheter or other devices may be easily used. For example, a graphical display may be used to conveniently present a representation of the sensing elements arrayed on the electrode tip to help see which portion of the electrode is seeing what signal (e.g., whether the signal is for position or contact sensing, or for evaluating the properties of the tissue itself). For example, the multiple sensing elements 1 shown in FIG. 1, may be presented graphically as three (3) independent annular arrays or rings on a “bulls eye” style graphic. The distal most elements may be represented in one of each quadrant of a most inner circle of the graphic, the middle set of elements may be represented in a middle ring, and the most proximal set of elements may be represented by an outer ring, e.g., staggered relative to the preceding ring. Moreover, the signal processing may be completed in an intuitive manner to correlate a color in the graphic to a given proximity to tissue (e.g., green for fully opposed, yellow for close, red for distant, and the like). Additionally, as previously described, an electrical anatomical map may be constructed using the position and heartbeat information derived and displayed in an intuitive manner.

Furthermore, other elements may be helpful in constructing the sensing and illumination hardware on the proximal end 4a of the catheter 4 or other device, including lenses to focus or direct illumination, and/or focus and/or direct the captured signals to be sensed by the signal sensing element(s). Moreover, filters may be used to narrow the spectrum of illumination and/or the collected, measured, or captured signals.

It will also be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. An apparatus for performing a procedure within a patient's body, comprising:
a tubular member comprising a proximal end, a distal end sized for introduction into a patient's body, and one or more lumens extending between the proximal and distal ends;
a distal tip on the distal end for contacting tissue; and
one or more optical elements on the distal tip configured to transmit illumination beyond the distal tip and capture optical signals from tissue or fluids adjacent the distal tip.

2. The apparatus of claim 1, further comprising one or more processors coupled to the one or more optical sensors to determine at least one of proximity to tissue and contact with tissue based on the optical signals.

3. The apparatus of claim 1, wherein the distal tip comprises an electrode.

4. The apparatus of claim 1, wherein the one or more optical elements comprise a plurality of optical elements arranged on the distal tip in one or more arrays.

5. The apparatus of claim 4, wherein the plurality of optical elements are arranged on the distal tip in a plurality of annular arrays.

6. The apparatus of claim 1, wherein each optical element comprises an optical fiber that transmits light from an illumination source coupled to the proximal end of the tubular member to the distal tip.

7. The apparatus of claim 1, wherein each optical element comprises an optical fiber that transmits optical signals from the distal tip to one or more processors coupled to the proximal end of the tubular member.

8. The apparatus of claim 1, wherein each optical element comprises a light source at the distal tip.

9. The apparatus of claim 1, wherein each optical element comprises a detector at the distal tip.

10. The apparatus of claim 1, wherein the one or more optical elements comprise one or more sources of one of visible light, infrared light, and ultraviolet light.

11. An apparatus that senses tissue proximity that includes one or more optical illumination and capturing elements, and one or more capturing sensors.

12. The apparatus of claim 1, wherein each individual optical illumination and capturing element comprises a glass fiber.

13. The apparatus of claim 11, wherein each optical illumination and capturing element comprises an illumination source configured to transmit light at one or more wavelengths to induce auto-fluorescence of cardiac tissue.

14. The apparatus of claim 13, where the capturing sensor is a camera.

15. The apparatus of claim 11, wherein the one or more optical illumination and capturing elements comprise a single CMOS or CCD array for capturing optical signals.

16. The apparatus of claim 11, wherein the one or more optical illumination and capturing elements comprise one or more optical capturing elements, and one or more photo diodes for illumination.

17. The apparatus of claim 16, wherein the number of capturing elements to the number of photo diodes is one-to-one.

18. A method for performing a procedure within a patient's body, comprising:
introducing a distal end of a tubular member into a patient's body;
placing a distal tip within a body lumen of the patient's body in contact with or in proximity to tissue adjacent the body lumen;
directing illumination from the distal tip towards the tissue;
acquiring optical signals corresponding to light reflect towards the distal tip within the body lumen; and
analyzing the optical signals to determine the proximity of the distal tip relative to the tissue.

19. The method of claim 18, wherein the body lumen comprises a chamber of the patient's heart.

20. The method of claim 18, wherein the optical signals are analyzed before performing ablation on a wall of the heart.
